(19) United States
(12) Patent Application Publication
(10) Pub. No.: US 2024/0334581 A1
(43) Pub. Date: Oct. 3, 2024

(54) CONTROL DEVICE, OBSERVATION SYSTEM, AND CONTROL METHOD

(71) Applicant: Sony Olympus Medical Solutions Inc., Tokyo (JP)

(72) Inventors: Shutaro YOROZU, Tokyo (JP); Shunsuke HAYASHI, Tokyo (JP)

(73) Assignee: Sony Olympus Medical Solutions Inc., Tokyo (JP)

(21) Appl. No.: 18/596,689

(22) Filed: Mar. 6, 2024

(30) Foreign Application Priority Data
Mar. 27, 2023 (JP) 2023-050295

(51) Int. Cl. H05B 47/28; A61B 1/00; A61B 1/06

(52) U.S. Cl. CPC H05B 47/28; A61B 1/0004; A61B 1/00045; A61B 1/00055; A61B 1/0655

(57) ABSTRACT

A technology advantageous for observing an observation target in a state where the observation target is irradiated with light emitted under an appropriate temperature condition in a light source device is provided. A control device includes a temperature information acquisition unit that acquires light source temperature information that is information regarding a temperature of a light source device that emits light with which biological tissues are to be irradiated, and a control unit that controls a notification on the basis of the light source temperature information.

### CROSS-REFERENCE TO RELATED APPLICATION

This application claims the benefit of Japanese Priority Patent Application JP 2023-050295 filed Mar. 27, 2023, the entire content of which is incorporated herein by reference.

### TECHNICAL FIELD

The present disclosure relates to a control device, an observation system, and a control method.

### BACKGROUND ART

In a medical field such as surgery and clinical research, imaging is performed while irradiating biological tissues with light in order to observe characteristics and a state of the biological tissues using observation equipment such as an endoscope and a microscope. In an endoscope device disclosed in PTL 1, RGB laser light emitted from a light source unit under the control of a light source control unit is guided to an illumination port by a light guide and is emitted from the illumination port toward an observation area in a body cavity.

### CITATION LIST

### Patent Literature

[PTL 1]
JP 2020-98604A

### SUMMARY

### Technical Problem

There is a case where it is necessary for a light source that emits light with which an observation target is to be irradiated to have an appropriate operating temperature in order to stably emit desired light. In a case where such a suitable operating temperature is not an ordinary temperature, the light source immediately after activation does not have a suitable operating temperature, and thus is not able to stably emit desired light.

In other words, a corresponding preparation period is necessary for adjusting a temperature of the light source until the light source can stably emit desired light after activation. Light to be emitted by a light source that is not ready does not have desired characteristics, and a captured image obtained by imaging an observation target irradiated with such light includes the observation target which is imaged under an inappropriate light irradiation condition.

On the other hand, it is not easy for a user to grasp whether or not the activated light source has an appropriate operating temperature (that is, whether or not the light source is ready). It is therefore not easy for the user to appropriately determine an observation start timing, and there is a concern that the observation target may be observed under an inappropriate light irradiation condition.

The present disclosure provides a technology advantageous for observing an observation target in a state where the observation target is irradiated with light emitted under an appropriate temperature condition in a light source device.

### Solution to Problem

One aspect of the present disclosure relates to a control device including a temperature information acquisition unit that acquires light source temperature information that is information regarding a temperature of a light source device that emits light with which biological tissues are to be irradiated, and a control unit that controls a notification on the basis of the light source temperature information.

The notification may include a notification regarding a state of the light source device.

The notification may include a notification regarding a use preparation state of the light source device. The notification may include a notification regarding availability of a function using the light source device. The notification may include a notification regarding whether or not switching between a plurality of modes having different types of light to be emitted from the light source device is possible.

The notification may include a notification regarding availability of the light source device.

The notification may include a notification by display on a display.

The notification by display on the display may include a notification to be made by activating or deactivating a software key displayed on the display.

The notification by display on the display may include a notification to be displayed on the display by a software key displayed on the display being selected.

The display may be a touch panel display.

The notification may include a notification by sound.

The control device may further include a notification unit that makes the notification.

The control device may be provided separately from a notification unit that makes the notification.

The light source temperature information may include information regarding the temperature of the light source device.

The light source temperature information may include information as to whether or not the temperature of the light source device is within an allowable temperature range.

The control unit may control the notification on the basis of a state of the light source device evaluated by comparing the light source temperature information acquired by the temperature information acquisition unit with reference data of the light source temperature information.

The control unit may control a temperature adjustment unit that adjusts the temperature of the light source device.

The control unit may control the temperature adjustment unit to individually adjust temperatures of a plurality of light sources included in the light source device. The control unit may control the temperature adjustment unit to collectively adjust temperatures of a plurality of light sources included in the light source device.

The light to be emitted from the light source device may include visible light.

The light to be emitted from the light source device may include excitation light that excites a specific fluorescent substance.

Another aspect of the present disclosure relates to an observation system including a light source device that emits light with which biological tissues are to be irradiated, and a control device, in which the control device includes a temperature information acquisition unit that acquires light source temperature information that is information regarding a temperature of the light source device; and a control unit that controls a notification on the basis of the light source temperature information.

The observation system may further include an imaging device that acquires an image of the biological tissue and a display device that displays the image.

Another aspect of the present disclosure relates to a control method including acquiring light source temperature information that is information regarding a temperature of a light source device that emits light with which biological tissues are to be irradiated, and controlling a notification on the basis of the light source temperature information.

### BRIEF DESCRIPTION OF DRAWINGS

FIG. 1 is a view illustrating an example of a medical observation system and particularly illustrates a case where the medical observation system is configured as an endoscope device including a rigid endoscope (endoscope).

FIG. 2 is a block diagram illustrating functional configuration examples of an imaging device, a control device, a light source device, and a display device.

FIG. 3 is a view illustrating an example of a user interface of the control device.

FIG. 4 is a view illustrating an example of the display device.

FIG. 5 is a view illustrating a first notification form example in the user interface.

FIG. 6 is a view illustrating the first notification form example in the user interface.

FIG. 7 is a view illustrating the first notification form example in the user interface.

FIG. 8 is a view illustrating a second notification form example in the user interface.

FIG. 9 is a view illustrating the second notification form example in the user interface.

FIG. 10 is a view illustrating a third notification form example in the user interface.

FIG. 11 is a view illustrating the third notification form example in the user interface.

FIG. 12 is a view illustrating a fourth notification form example in the user interface.

FIG. 13 is a view illustrating the fourth notification form example in the user interface.

FIG. 14 is a view illustrating the fourth notification form example in the user interface.

FIG. 15 is a view illustrating a fifth notification form example in the user interface.

FIG. 16 is a view illustrating the fifth notification form example in the user interface.

FIG. 17 is a view illustrating the fifth notification form example in the user interface.

FIG. 18 is a view illustrating the fifth notification form example in the user interface.

FIG. 19 is a view illustrating an example of mode switching flow in the medical observation system.

FIG. 20 is a conceptual view illustrating an example of a medical observation system configured as an operative field illumination observation device in which a ring light (open field illumination device for living body observation) is connected to a light source device.

FIG. 21 is a view illustrating an example of a medical observation system configured as a microscope system.

### DESCRIPTION OF EMBODIMENTS

Hereinafter, an embodiment of the present disclosure will be described with reference to the drawings.

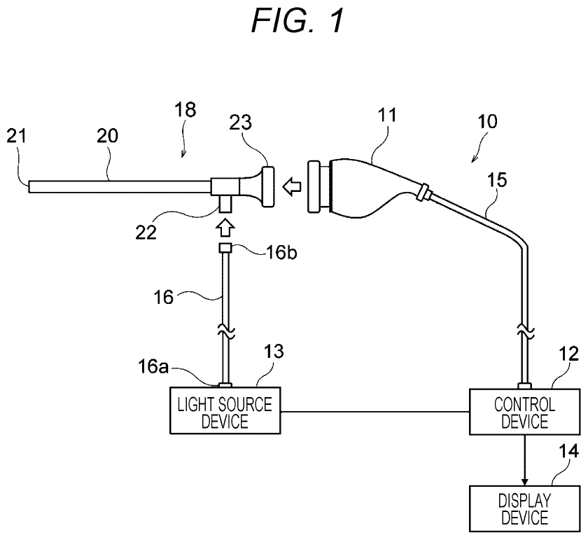

FIG. 1 is a view illustrating an example of a medical observation system 10 and particularly illustrates a case where the medical observation system 10 is configured as an endoscope device including a rigid endoscope (endoscope) 18.

The medical observation system 10 is used to observe a target site that is an observation target (subject) via a captured image or to observe the target site with the naked eye. The term “medical” as used herein is interpreted in a broad sense and is concept that can include not only treatment of diseases and injuries but also various actions for the purpose of maintaining, recovering, or promoting health, and the concept of “medical” also includes an action mainly for the purpose of research.

The medical observation system 10 illustrated in FIG. 1 includes an imaging device 11, a control device 12, a light source device 13, and a display device 14. The light source device 13 is provided to be connectable to the rigid endoscope 18 and emits light under the control of the control device 12. The light source device 13 can emit light in an arbitrary wavelength range and can employ an arbitrary device configuration capable of emitting broadband light such as white light and/or narrow band light, for example.

The broadband light mentioned here is light having a wide wavelength band, but specific spectral characteristics (wavelength distribution) are not limited, and in particular, white light is light that contains visible light components of various colors and can be perceived as white. The narrow band light includes light in a specific wavelength region of the visible light wavelength region and the invisible light wavelength region as a main light component and has arbitrary spectral characteristics based on a center wavelength (peak wavelength).

The rigid endoscope 18 of the present example is connected to the light source device 13 via a detachable light guide 16. In other words, a first light guide end portion 16a on one end side of the light guide 16 is detachably attached to the light source device 13, and a second light guide end portion 16b on the other end side is detachably connected to an optical connection portion 22 of the rigid endoscope 18.

The rigid endoscope 18 illustrated in FIG. 1 includes an insertion portion 20, and the optical connection portion 22 and an imaging connection portion 23 provided on a proximal end side of the insertion portion 20. A light transmission portion (light guide) and an objective lens are provided on an end surface of an insertion distal end portion 21 of the insertion portion 20 located on the side opposite to the proximal end side. The light transmitted from the light source device 13 through the light guide 16 is emitted from the light transmission portion on the end surface of the distal end side of the insertion portion 20. Then, observation light that is light (for example, reflected light or fluorescence) from the observation target enters the objective lens and is guided to the imaging connection portion 23 through the inside of the insertion portion 20.

The imaging connection portion 23 is detachably connected to a connection portion of the imaging device 11. The observation light transmitted through the objective lens is incident on the imaging device 11 through the imaging connection portion 23 and received by the imaging device 11. The imaging connection portion 23 can also function as an eyepiece unit. In a state where the imaging connection portion 23 is detached from the imaging device 11, a user such as a doctor can directly view the observation light via the imaging connection portion 23. The imaging device 11 is provided to be connectable to the rigid endoscope 18, receives observation light via the rigid endoscope 18 connected thereto and captures and acquires an image of the observation target (biological tissues). The imaging device 11 is connected to the control device 12 via a signal transmission cable 15. Data (that is, the captured image signal) of the captured image corresponding to the observation light received through the rigid endoscope 18 is transmitted from the imaging device 11 to the control device 12 through the signal transmission cable 15.

The control device 12 is connected to the imaging device 11, the light source device 13, and the display device 14, and controls the imaging device 11, the light source device 13, and the display device 14. Furthermore, the control device 12 can also control the rigid endoscope 18 connected to the imaging device 11 via the imaging device 11. For example, the control device 12 causes the display device 14 to display a captured image acquired by the imaging device 11 and controls light emission of the light source device 13.

In a case where the rigid endoscope 18 is used in the medical observation system 10 described above, for example, the insertion portion 20 of the rigid endoscope 18 is inserted into the abdominal cavity inside the peritoneum to be observed, and light is emitted from the insertion distal end portion 21 in the abdominal cavity. As described above, the biological tissues that are the observation target are irradiated with the light emitted from the light source device 13 and emitted from the rigid endoscope 18.

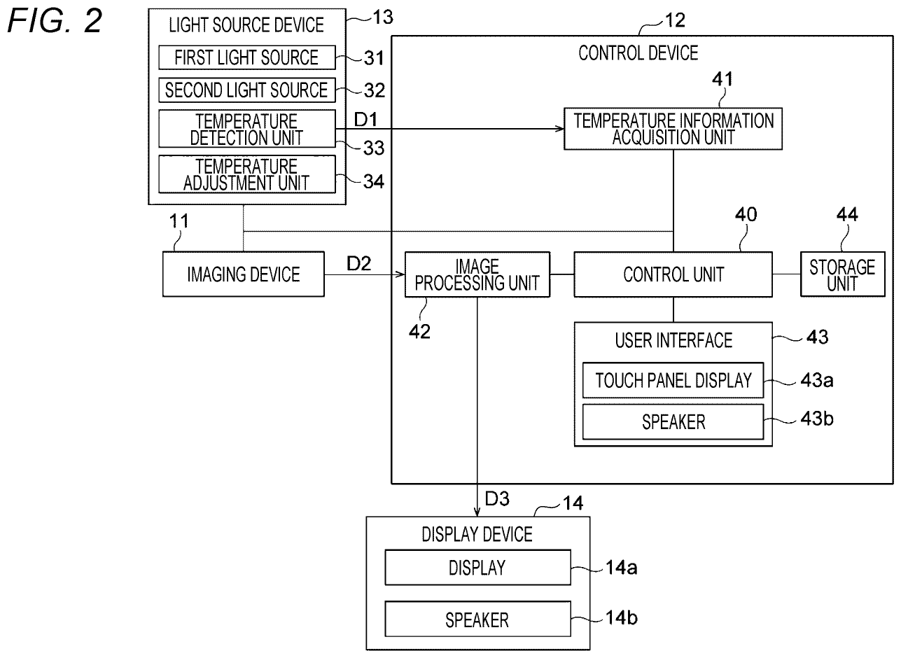

FIG. 2 is a block diagram illustrating functional configuration examples of the imaging device 11, the control device 12, the light source device 13, and the display device 14.

The light source device 13 illustrated in FIG. 2 includes a first light source 31, a second light source 32, a temperature detection unit 33, and a temperature adjustment unit 34.

The first light source 31 and the second light source 32 emit light with which the observation target is to be irradiated under the control of the control device 12 (particularly, the control unit 40) and can be constituted with an arbitrary light source device (for example, a xenon lamp, a light-emitting diode (LED), or a laser).

The light to be emitted from the first light source 31 and the second light source 32 is not limited and may include light in a visible light wavelength band (that is, visible light) or may include light in a wavelength band outside the visible light wavelength band (that is, invisible light). In particular, the light to be emitted from the first light source 31 and the second light source 32 may include broadband light such as white light for brightly illuminating the observation target or may include narrow band light such as excitation light for exciting a specific fluorescent substance that can be possessed by the observation target or other special light.

The specific fluorescent substance that can be possessed by the observation target may be a drug or a fluorescent dye applied to the observation target or may be a fluorescent substance constituting the observation target itself.

The light source device 13 includes two light sources 31 and 32 in the example illustrated in FIG. 2, but may include three or more light sources or may include a single light source.

The temperature detection unit 33 measures and acquires temperatures of the light sources (the first light source 31 and the second light source 32 in the example illustrated in FIG. 2) included in the light source device 13 under the control of the control device 12 (particularly, the control unit 40). Then, the temperature detection unit 33 transmits light source temperature information D1 based on the measured temperatures of the light sources 31 and 32 to the control device 12 (particularly, the temperature information acquisition unit 41). A temperature sensor of the temperature detection unit 33 that measures the temperatures of the light sources can be configured by an arbitrary sensor device (for example, a thermistor). The temperature detection unit 33 may evaluate temperature states of the light sources 31 and 32 on the basis of the measured temperatures of the light sources 31 and 32 or does not have to evaluate such temperature states of the light sources 31 and 32. In a case where the temperature states of the light sources 31 and 32 are evaluated, the temperature detection unit 33 may transmit the light source temperature information D1 including the evaluation results of the temperature states of the light sources 31 and 32 to the control device 12. On the other hand, in a case where the temperature states of the light sources 31 and 32 are not evaluated by the temperature detection unit 33, only the information of the measured temperatures of the light sources 31 and 32 may be included in the light source temperature information D1, or the measured temperatures of the light sources 31 and 32 and other information may be included in the light source temperature information D1.

The information indicating the evaluation results of the temperature states of the light sources 31 and 32 may include, for example, information indicating whether or not the temperatures of the light sources 31 and 32 of the light source device 13 are within allowable temperature ranges. The allowable temperature ranges mentioned here are temperature ranges in which desired light can be stably emitted in the light sources 31 and 32 and are determined in accordance with a device configuration of the light sources 31 and 32 and the characteristics of the desired light.

For example, in a case where there is an optimum temperature for emitting desired light for each of the light sources 31 and 32, a temperature range determined based on a target temperature (for example, a temperature range of “target temperature±α° C.”) can be set as the allowable temperature range while the optimum temperature is set as the target temperature for the temperature adjustment of each of the light sources. Here, “α” may be variably determined on the basis of various circumstances and conditions. The allowable temperature range may be a temperature range of “target temperature-β° C.” to “target temperature±α° C.” (where “α≠β”). Furthermore, the temperature detection unit 33 may include other information for temperature adjustment of the light source device 13 in the light source temperature information D1. The other information for the temperature adjustment of the light source device 13 may include, for example, information indicating the allowable temperature ranges of the light sources 31 and 32, information indicating a duration in which the temperatures of the light sources 31 and 32 are kept in the allowable temperature ranges, and information indicating temporal temperature changes of the light sources 31 and 32.

The temperature adjustment unit 34 adjusts the temperatures of the light sources 31 and 32 of the light source device 13 under the control of the control device 12 (particularly, the control unit 40). The temperature adjustment unit 34 can employ any device configuration for adjusting the temperatures of the light sources 31 and 32 and can include a heating device for increasing the temperatures of the light sources 31 and 32 and/or a cooling device for decreasing the temperatures of the light sources 31 and 32.

Generally, there is a possibility that the temperatures of the light sources 31 and 32 may gradually rise, and the light sources 31 and 32 may be overheated beyond the target temperature by energization. Thus, the temperature adjustment unit 34 may be provided as a cooling device capable of cooling the light sources 31 and 32 in order to prevent the light sources 31 and 32 from being overheated. The cooling device is capable of cooling the light sources 31 and 32 in any configuration and in any manner. For example, the cooling device can include a device such as a Peltier element that actively cools the light sources 31 and 32 by energization or a device such as a heat sink that structurally promotes heat dissipation.

The control device 12 illustrated in FIG. 2 includes a control unit 40, a temperature information acquisition unit 41, an image processing unit 42, a user interface 43, and a storage unit 44.

Under the control of the control unit 40, the temperature information acquisition unit 41 acquires the light source temperature information D1, which is information regarding the temperature of the light source device 13, from the temperature detection unit 33 of the light source device 13 and transmits the light source temperature information D1 to the control unit 40. Although specific information included in the light source temperature information D1 is not limited, in the present example, information for the temperature adjustment of the light sources 31 and 32 is included in the light source temperature information D1. As described above, the light source temperature information D1 may include the measured temperatures of the light sources 31 and 32 or may include information derived from the measured temperatures of the light sources 31 and 32. Under the control of the control unit 40, the image processing unit 42 performs image processing on the captured image data D2 transmitted from the imaging device 11 to generate output image data D3 and transmits the output image data D3 to the display device 14 or to the user interface 43 via the control unit 40.

The output image data D3 is image data that can be presented to the user via the display and may include image data in various display forms. For example, the output image data D3 may include data of individual captured images of the observation target generated on the basis of the individual captured image data D2. In addition, the output image data D3 may include data of an image (for example, an image in which a plurality of captured images is superimposed) generated on the basis of a combination of the plurality of pieces of captured image data D2.

The user interface 43 includes a touch panel display 43a and a speaker 43b that function under the control of the control unit 40.

The touch panel display 43a functions as an input unit that receives an input from the user and also functions as a notification unit that notifies the user of various kinds of display information (for example, image information (including character image information)).

For example, the output image data D3 transmitted via the control unit 40 is displayed on the touch panel display 43a under the control of the control unit 40. The speaker 43b functions as a notification unit that notifies the user of various kinds of sound information.

In the example illustrated in FIG. 2, the display device 14 including a display 14a and a speaker 14b also functions as a notification unit. For example, the control unit 40 can cause the output image data D3 transmitted from the image processing unit 42 to the display device 14 to be displayed on the display 14a of the display device 14 and can cause the sound information to be emitted from the speaker 14b.

As described above, in the example illustrated in FIG. 2, the notification unit which is controlled by the control unit 40 on the basis of the light source temperature information D1 and which makes a notification is provided in the control device 12 itself and is provided in the display device 14 separate from the control device 12. The storage unit 44 stores various kinds of data under the control of the control unit 40, and the control unit 40 reads the various kinds of data from the storage unit 44.

The data stored in the storage unit 44 is not limited, and image data such as the captured image data D2 and the output image data D3 may be stored in the storage unit 44, or programs and various kinds of data necessary for control processing of the control unit 40 may be stored in the storage unit 44. In addition, various kinds of data to be read from the storage unit 44 can be appropriately used for an arbitrary purpose.

Note that the storage unit 44 of the present example is provided as part of the control device 12, but the storage unit 44 may be provided in a device (for example, the light source device 13, a server, or an external storage device such as a USB memory) separate from the control device 12. In this case, the control unit 40 stores and reads various kinds of data in and from the storage unit 44 in a wired and/or wireless manner. Further, under the control of the control unit 40, various kinds of data may be transmitted and received between the storage unit 44 in the control device 12 and a storage unit separate from the control device 12. In a case where the light source (that is, the light) to be used in the medical observation system 10 is not changed, it may not be necessary to update “various kinds of data regarding the light source” stored in the storage unit 44. On the other hand, in a case where it is necessary to update the “various kinds of data regarding the light source” stored in the storage unit 44, update data may be stored and used in the storage unit 44 of the control device 12 from a storage unit separate from the control device 12. In this case, even in a case where a new light source (that is, new light) is used in the medical observation system 10, it is possible to appropriately observe the observation target using such a new light source.

The control unit 40 controls the temperature adjustment unit 34 on the basis of the light source temperature information D1 acquired via the temperature information acquisition unit 41.

As described above, each of the light sources 31 and 32 has a temperature range (that is, an allowable temperature range) in which desired light can be stably emitted. The control unit 40 controls the temperature adjustment unit 34 on the basis of the light source temperature information D1 to adjust the temperature of each of the light sources 31 and 32 to an allowable temperature range.

A specific manner of the temperature control of each of the light sources 31 and 32 by the control unit 40 is not limited, and for example, feedback control or feedforward control may be performed. Furthermore, the control unit 40 may adjust the temperature of each of the light sources 31 and 32 by controlling the temperature adjustment unit 34 in consideration of information such as the life and wavelength shift of each of the light sources 31 and 32 as a device.

In a case where the light source device 13 includes a plurality of light sources 31 and 32 as in the illustrated example, the control unit 40 controls the temperature adjustment unit 34 so that the temperatures of all of the plurality of light sources 31 and 32 are appropriately adjusted. However, a specific manner of temperature adjustment of the plurality of light sources 31 and 32 is not limited.

As an example, the control unit 40 may control the temperature adjustment unit 34 so as to individually adjust the temperatures of the plurality of light sources 31 and 32 of the light source device 13. For example, in white light observation, in a case where the light source device 13 includes a plurality of light sources that emits a plurality of types of visible light (for example, RGB light) having different wavelength bands, the target temperatures and the allowable temperature ranges of the respective light sources may be different from each other. Similarly in the fluorescence observation, in a case where the light source device 13 includes a plurality of light sources that emits a plurality of types of excitation light having different wavelength bands, the target temperatures and the allowable temperature ranges of the respective light sources may be different from each other.

As described above, even in a case where the light source device 13 includes a plurality of light sources having different target temperatures and allowable temperature ranges, the temperature adjustment unit 34 individually adjusts the temperatures of the light sources, so that each light source can emit desired light. As a result, an image of the observation target is captured under an appropriate light irradiation condition, and an appropriate captured image is obtained. In addition, by performing temperature adjustment for each light source, it can be expected that the life of each light source is prolonged.

Furthermore, the control unit 40 may control the temperature adjustment unit 34 so as to collectively adjust the temperatures of the plurality of light sources 31 and 32 of the light source device 13. In other words, the temperatures of the plurality of light sources 31 and 32 may be collectively adjusted so that the plurality of light sources 31 and 32 basically has the same temperature. In this case, it is not necessary to prepare a temperature adjustment device for each light source, and the temperature adjustment unit 34 can adjust the temperatures of the plurality of light sources 31 and 32 using a common temperature adjustment device. Thus, the device configuration of the temperature adjustment unit 34 can be simplified and downsized, and the temperature adjustment unit 34 can be configured at low cost.

Furthermore, the control unit 40 controls the notification in the notification unit (the user interface 43 and the display device 14 in the example illustrated in FIG. 2) on the basis of the light source temperature information D1. In other words, the control unit 40 executes a control method including acquiring the light source temperature information D1 that is information regarding the temperature of the light source device 13 that emits light with which the observation target is to be irradiated, and controlling the notification on the basis of the light source temperature information D1. Such a notification via the notification units 14 and 43 may include a notification regarding a state of the light source device 13 (for example, light sources 31 and 32) and may include, for example, a notification regarding temperature adjustment states or a notification regarding use preparation states of the light sources 31 and 32 of the light source device 13.

For example, before the user operates the medical observation system 10 (for example, the control device 12), the notification units 14 and 43 can make a notification of a preparation status of the light source device 13 (for example, whether the light source device 13 is ready or not ready). On the other hand, even though the user operates the control device 12 to input an execution instruction in a certain observation mode to the control unit 40, there is a case where the light source device 13 (for example, a corresponding light source) to be used in the observation mode may not be ready. In such a case, the notification units 14 and 43 can notify the user that the observation mode is not executable.

As an example, the control unit 40 may control the notification via the notification units 14 and 43 on the basis of the state of the light source device 13 evaluated by comparing the light source temperature information D1 acquired by the temperature information acquisition unit 41 with reference data of the light source temperature information. The “reference data of the light source temperature information” mentioned here may be in the form of a data table, may be in any other data form, may be held by the control unit 40, or may be stored in a memory (for example, the storage unit 44) readable by the control unit 40.

Note that the temperature states of the light sources 31 and 32 may be evaluated by the control unit 40 in this manner, but may be evaluated by the light source device 13 (the temperature detection unit 33 in the example illustrated in FIG. 2) as described above. In a case where the light source device 13 evaluates the temperature states of the light sources 31 and 32, it is possible to reduce load of processing for notification control in the control unit 40.

The control unit 40 can make various notifications to the user via the notification units 14 and 43, but specific content of the notifications is not limited. For example, instead of notifying the user that the observation mode selected by the user is not executable, a notification may be provided to notify the user that it is necessary to wait until the device (for example, the light source device 13) is ready.

Furthermore, such a notification via the notification units 14 and 43 may include a notification regarding availability of the light source device 13. For example, while the temperatures of the light sources 31 and 32 of the light source device 13 are within the allowable temperature ranges, a notification for notifying the user that the light source device 13 is available may be made. On the other hand, while the temperatures of the light sources 31 and 32 of the light source device 13 are out of the allowable temperature ranges, a notification to notify the user that the light source device 13 is not available may be made.

Furthermore, such a notification via the notification units 14 and 43 may include a notification regarding availability of a function using the light source device 13.

Furthermore, such a notification via the notification units 14 and 43 may include a notification regarding whether or not switching between a plurality of modes having different types of light to be emitted from the light source device 13 is possible. For example, while a temperature of the light source that emits desired light (for example, white light or excitation light) to be used in a desired mode is within an allowable temperature range, a notification to notify the user that switching to the desired mode is possible may be made. On the other hand, while a temperature of the light source that emits desired light to be used in a desired mode is out of the allowable temperature range, a notification to notify the user that the mode is not switchable to the desired mode may be made.

### (Notification Unit)

As described above, the user such as a doctor and an operation assistant can accurately recognize a state (for example, temperature adjustment states or use preparation states of the light sources 31 and 32, and the like) of the light source device 13 in a timely manner by the notification via the notification unit (particularly, the notification to be controlled by the control unit 40). In the example illustrated in FIG. 2, the user interface 43 of the control device 12 and the display device 14 are used as such a notification unit.

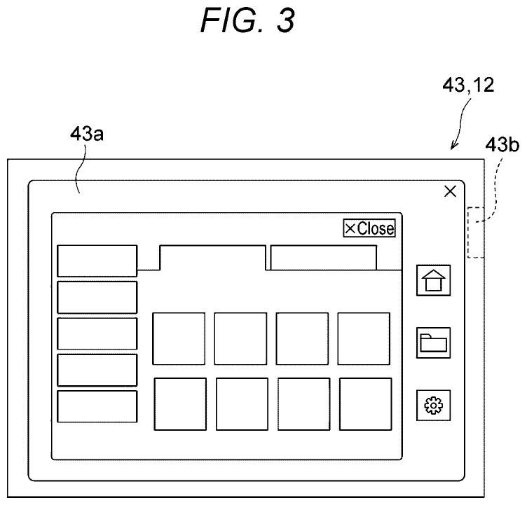

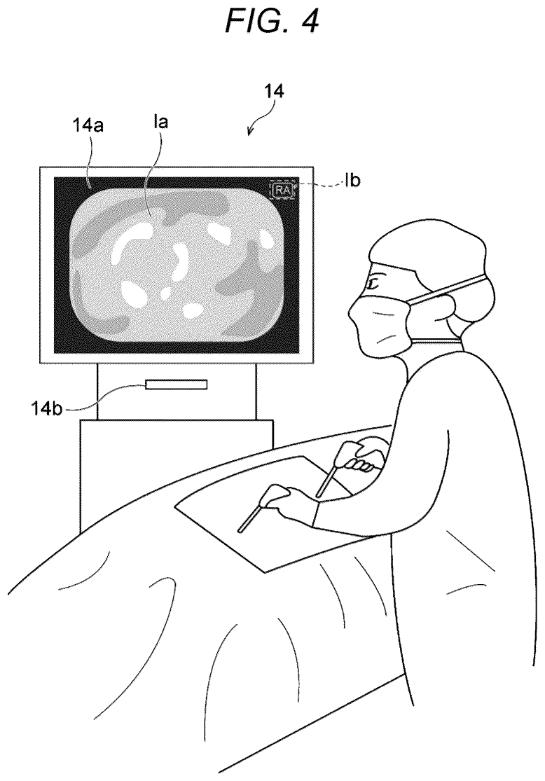

FIG. 3 is a view illustrating an example of the user interface 43 of the control device 12. FIG. 4 is a view illustrating an example of the display device 14. The display of the user interface 43 illustrated in FIG. 3 includes the touch panel display 43a functioning as an input unit and a notification unit and displays various software keys. The software keys to be displayed on the touch panel display 43a can be appropriately changed in accordance with various modes and situations under the control of the control unit 40 (see FIG. 2). In a case where a desired software key is selected, the user touches a corresponding portion of the touch panel display 43a.

On the other hand, the display 14a of the display device 14 illustrated in FIG. 4 functions as a notification unit that performs only display of the display information and does not function as an input unit that receives operation from the user. Thus, the user basically does not need to touch the display 14a of the display device 14.

In general, a doctor who can come in contact with the observation target does not operate the control device 12 (for example, the touch panel display 43a as illustrated in FIG. 3) that may be contaminated, and operation of the control device 12 is performed by an operation assistant different from the doctor. In particular, the touch panel display 43a of the control device 12 is usually not installed near the observation target and has a small display area in many cases, and thus, it is not suitable as a display that the doctor views while performing medical practice. Thus, the doctor generally performs medical practice while checking the display device 14 (see FIG. 4) that is provided separately from the control device 12 and can display the captured image Ia of the observation target in a large size.

Thus, according to the example illustrated in FIG. 2 in which the user interface 43 of the control device 12 and the display device 14 are used as the notification unit, it is possible to appropriately and quickly notify both the doctor and the operation assistant engaged in the operation of the medical observation system 10.

In other words, the operation assistant who performs operation such as setting of the medical observation system 10 via the touch panel display 43a of the control device 12 can quickly recognize the notification via the user interface 43 of the control device 12. On the other hand, the doctor who performs medical practice such as insertion of the rigid endoscope 18 into the observation target can quickly recognize the notification via the display device 14.

Note that, on the display 14a of the display device 14, a position at which an image related to the notification (hereinafter referred to as a “notification image Ib”) is displayed and a display form are not limited. For example, as illustrated in FIG. 4, in a case where there is a region (such as, for example, a mask image region) in which the captured image Ia of the observation target is not displayed in the display region of the display 14a, the notification image Ib may be displayed in such a region in which the captured image Ia is not displayed. Alternatively, the notification image Ib may be displayed on the display 14a so as to be superimposed on the captured image Ia of the observation target. In a case where the notification image Ib is displayed so as to be superimposed on the captured image Ia, the notification image Ib is preferably displayed so that valid information obtained from the captured image Ia is not impaired as much as possible. For example, the notification image Ib is displayed in the vicinity of an edge (for example, in the vicinity of a corner) of a display area of the display 14a or the notification image

Ib is displayed in a translucent manner so that a superimposed portion of the captured image Ia can be visually recognized, thereby reducing influence on the captured image Ia on the display 14a.

The notification via the notification units 14 and 43 may include not only a notification by display (display information) on the displays (the touch panel display 43a and the display 14a) but also a notification by sound (sound information) from the speakers 43b and 14b (see FIGS. 3 and 4).

Typically, the notification by display on the display can be executed by displaying or not displaying the image (notification image Ib) such as an icon directly or indirectly indicating the state of the light source device 13 on the display. The user can recognize the state of the light source device 13 in accordance with whether or not the notification image Ib is displayed on the display.

Further, the notification on the touch panel display 43a capable of displaying the software keys may include a notification to be made by activating (enabling) or deactivating (disabling) the software keys displayed on the touch panel display 43a. By switching a state of an ON/OFF button of the software key function between an enabled state and a disabled state in this manner, it is possible to prevent the software key function from being executed in a state where the corresponding light source is not ready.

The notification by display on the touch panel display 43a of the control device 12 may include a notification to be displayed on the touch panel display 43a by a software key displayed on the touch panel display 43a being selected by the user. In this way, by making the notification in conjunction with operation of the user selecting the ON/OFF button of the software key function, it is possible to make the notification while effectively calling the user's attention.

Hereinafter, a notification example in the user interface 43 of the control device 12 will be described. The notification in the user interface 43 to be made in each of the following notification form examples is appropriately made under the control of the control unit 40.

### First Notification Form Example

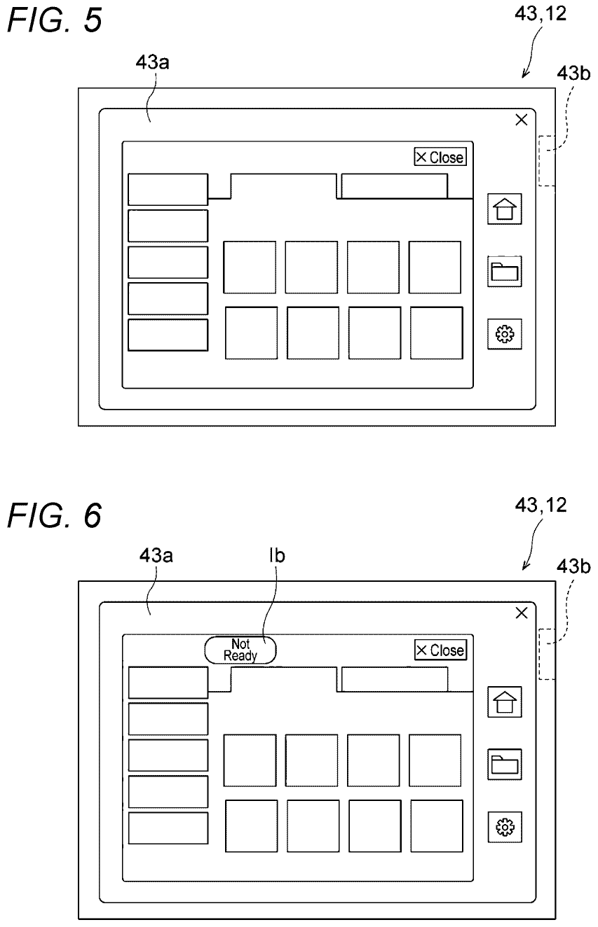

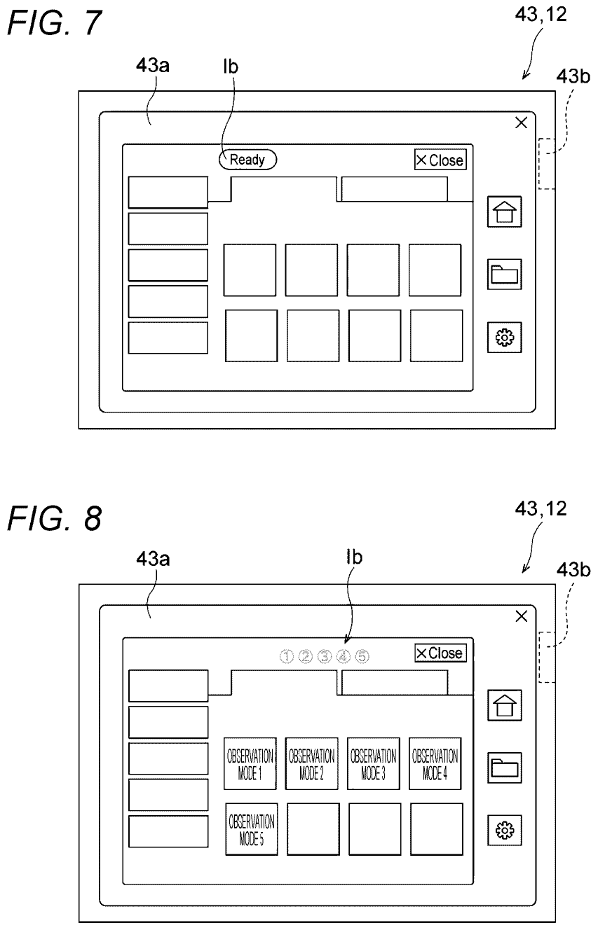

FIGS. 5, 6, and 7 are views for explaining a first notification form example in the user interface 43. FIG. 5 illustrates an example of display in a state where the light source device 13 (in particular, light sources 31 and 32) is not ready. FIG. 6 illustrates another example of the display in a state where the light source device 13 (in particular, light sources 31 and 32) is not ready. FIG. 7 illustrates an example of display in a state where the light source device 13 (in particular, light sources 31 and 32) is ready.

In the present notification form example, a notification that the light source device 13 is ready is made by displaying the corresponding notification image Ib (for example, an icon) on the touch panel display 43a. In other words, after the light source device 13 is ready, the notification image Ib indicating the ready state is displayed on the touch panel display 43a (see FIG. 7). On the other hand, while the light source device 13 is not ready, the related notification image Ib is not displayed on the touch panel display 43a (see FIG. 5), or the notification image Ib indicating the unready state is displayed on the touch panel display 43a (see FIG. 6).

According to the present notification form, the user can know the state of the light source device 13 by confirming the notification image Ib displayed on the touch panel display 43a or by confirming whether or not the notification image Ib is displayed on the touch panel display 43a.

In other words, while the notification image Ib is not displayed on the touch panel display 43a, the user can recognize that the light source device 13 is in the unready state (see FIG. 5). Alternatively, while the notification image Ib indicating the unready state is displayed on the touch panel display 43a, the user can recognize that the light source device 13 is in the unready state (see FIG. 6). Then, while the notification image Ib indicating the ready state is displayed on the touch panel display 43a, the user can recognize that the light source device 13 is in the ready state (see FIG. 7).

Note that the notification image Ib may be displayed on the touch panel display 43a only while the light source device 13 is in the unready state, and the notification image Ib does not have to be displayed on the touch panel display 43a while the light source device 13 is in the ready state. Also in this case, the user can recognize the state of the light source device 13 by checking whether or not the notification image Ib is displayed on the touch panel display 43a.

### Second Notification Form Example

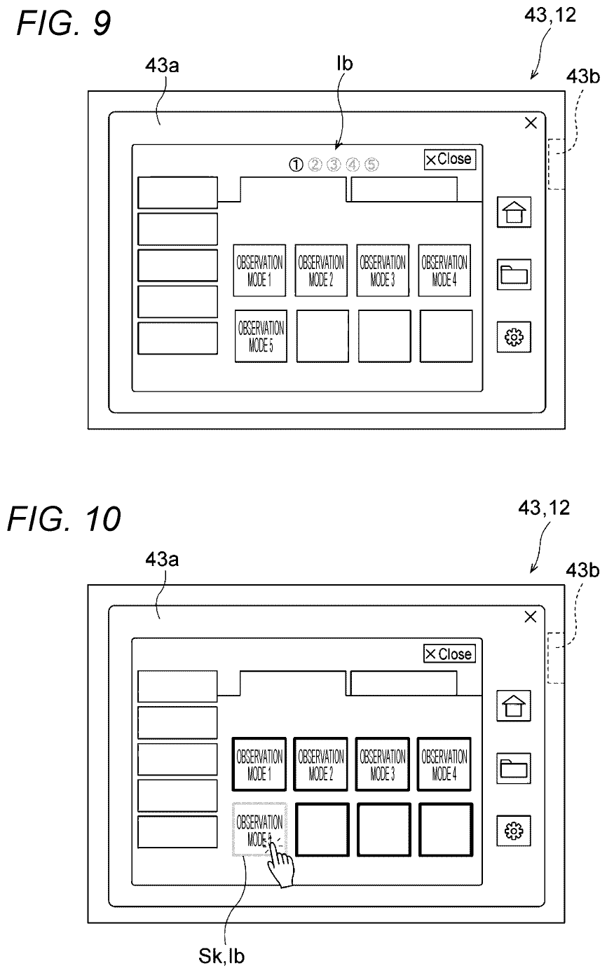

FIGS. 8 and 9 are views illustrating a second notification form example in the user interface 43. FIG. 8 illustrates an example of display in a state where the light source device 13 is not ready for all the observation modes 1 to 5. FIG. 9 illustrates an example of display in a state where the light source device 13 is ready for the observation mode 1, and the light source device 13 is not ready for the observation mode 2 to 5.

The notification in the present notification embodiment includes a notification regarding whether or not switching between a plurality of modes having different types of light to be emitted from the light source device 13 is possible.

In other words, in the present example, the observation mode can be switched between a plurality of modes (observation modes 1 to 5) in accordance with selection by the user. The types of light to be emitted from the light source device 13 (that is, light with which the observation target is to be irradiated) are different between the plurality of modes, and in particular, light sources that actually emit light (that is, light sources that emit light with which the observation target is to be irradiated) are different between two or more modes. The touch panel display 43a displays a plurality of notification images Ib (icons) corresponding to the respective modes, separately from the software keys (see notations of “observation mode 1”, and the like, in FIGS. 8 and 9) for selecting the respective modes. Then, the notification form of the notification image Ib corresponding to each mode on the touch panel display 43a is changed between in a case where the corresponding light source device 13 (in particular, light sources 31 and 32) is not ready and in a case where the corresponding light source device 13 is ready.

In other words, for each mode, while the corresponding light source is not ready (that is, during the unready state), the corresponding notification image Ib is grayed out on the touch panel display 43a (see FIG. 8). On the other hand, for each mode, while the corresponding light source is ready (that is, during the ready state), the corresponding notification image Ib is normally displayed (non-grayed-out display) on the touch panel display 43a (see FIG. 9).

The user can appropriately recognize switchable modes (that is, available modes) and unswitchable modes (that is, unavailable modes) by checking the notification image Ib displayed on the touch panel display 43a. By displaying both the switchable modes and the unswitchable modes on the touch panel display 43a in different notification forms, the user can easily and clearly grasp whether each mode is in an active state or an inactive state.

Note that the notification image Ib in the mode in which the corresponding light source is not ready is grayed out in the example illustrated in FIGS. 8 and 9, but may be displayed on the touch panel display 43a in any other form or does not have to be displayed on the touch panel display 43a.

### Third Notification Form Example

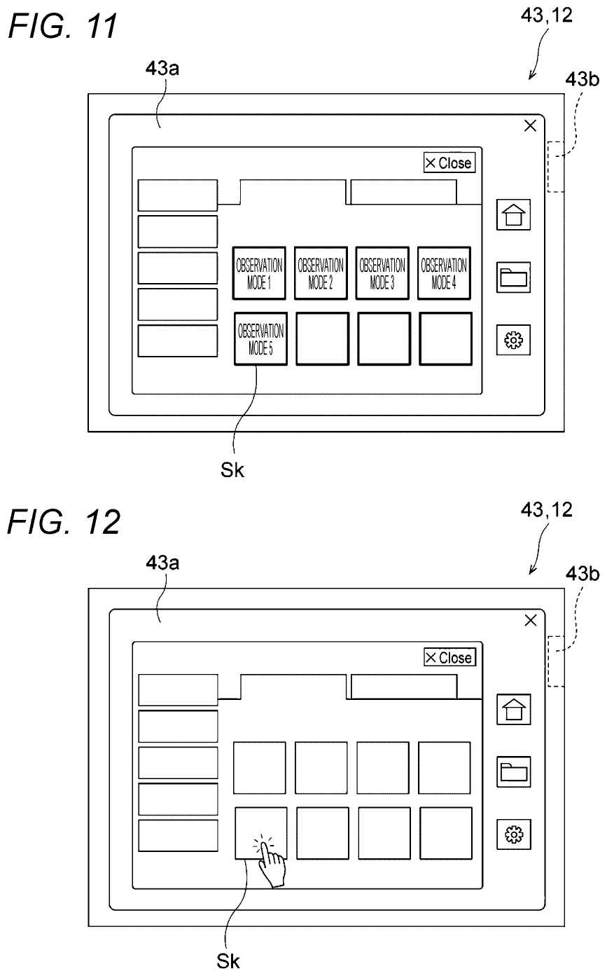

FIGS. 10 and 11 are views illustrating a third notification form example in the user interface 43. FIG. 10 illustrates an example of display in a state where the light source device 13 is ready for the observation mode 1 to 4 while the light source device 13 is not ready for the observation mode 5. FIG. 11 illustrates an example of display in a state where the light source device 13 is ready for all of the observation modes 1 to 5.

The notification in this notification form includes a notification to be made by activating or deactivating software keys displayed on the touch panel display 43a.

Also in the present example, similarly to the second notification form example described above, the observation mode can be switched among a plurality of modes (observation modes 1 to 5) in accordance with selection by the user, and the types of light to be emitted from the light source device 13 are different among the plurality of modes.

However, the present example is different from the second notification form example in that while in the second notification form example, the software key Sk and the notification image Ib related to each mode are separately displayed, in the present example, the software key Sk and the notification image Ib related to each mode are integrally displayed on the touch panel display 43a. In other words, the software key Sk for selection is displayed for each mode (observation mode 1 to 5), but the notification image Ib is displayed by the display image being changed between the software key Sk in the inactive state and the software key Sk in the active state. In the present example, display of the notification image Ib on the touch panel display 43a is implemented by graying out the software key Sk in the inactive state (see FIG. 10) while normally displaying the software key Sk in the active state (see FIG. 11). Note that the notification image Ib in the mode in which the corresponding light source is not ready may be displayed on the touch panel display 43a in a form other than grayed-out. Furthermore, as an example, display of the notification image Ib may be implemented by displaying only the software key Sk in the active state on the touch panel display 43a without displaying the software key Sk in the inactive state on the touch panel display 43a. However, by displaying both the mode in which the corresponding light source is not ready and the mode in which the corresponding light source is ready in notification forms different from each other, the user can easily and clearly grasp whether each mode is in the active state or the inactive state.

As in the present notification form example, by integrally displaying the software key Sk for mode selection and the notification image Ib, a display area of the touch panel display 43a can be efficiently used. In addition, the user can intuitively grasp whether each mode is in the active state or the inactive state.

### Fourth Notification Form Example

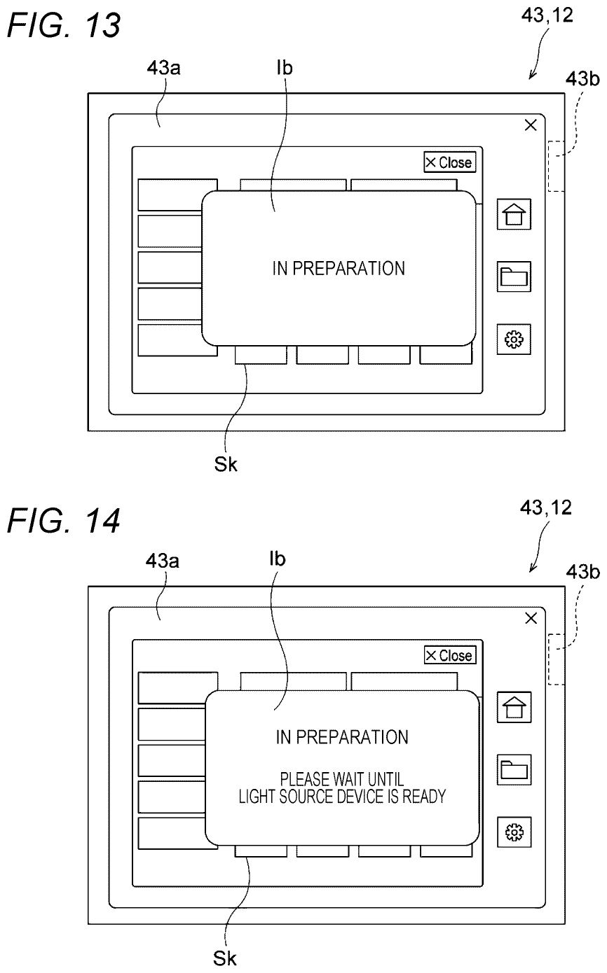

FIGS. 12 to 14 are views illustrating a fourth notification form example in the user interface 43. FIG. 12 illustrates an example of display immediately before a certain software key Sk is selected by the user. FIG. 13 illustrates an example of display immediately after a certain software key Sk is selected by the user. FIG. 14 illustrates another example of display immediately after a certain software key Sk is selected by the user. The notification in the present notification form includes a notification to be displayed on the touch panel display 43a by selecting the software key Sk displayed on the touch panel display 43a.

In other words, when the user selects the desired software key Sk (see FIG. 12), if the temperature of the corresponding light source of the mode associated with the selected software key Sk is in the allowable temperature range and ready, the notification image Ib is not displayed. In this case, normal display for executing the mode associated with the selected software key Sk is performed on the touch panel display 43a. On the other hand, in a case where the temperature of the corresponding light source in the mode associated with the selected software key Sk is out of the allowable temperature range and not ready, the notification image Ib is popped up on the touch panel display 43a as illustrated in FIGS. 13 and 14.

The notification image Ib displayed in this manner is not limited as long as it can notify the user that the corresponding light source of the mode associated with the selected software key Sk is not ready. For example, the notification image Ib may directly indicate only a fact that the selected mode is in preparation as illustrated in FIG. 13 or may also indicate a more specific reason as illustrated in FIG. 14.

By the user touching the corresponding portion of the touch panel display 43a to select a mode for deleting the pop-up display of the notification image Ib, the notification image Ib is deleted from the touch panel display 43a. In this case, the display on the touch panel display 43a returns to a state where the selection of the software key Sk is canceled (that is, a state before the software key Sk is selected). Note that while the temperature of the corresponding light source in the mode associated with the selected software key Sk is out of the allowable temperature range, the notification image Ib is repeatedly popped up in response to the touch operation by the user.

### Fifth Notification Form Example

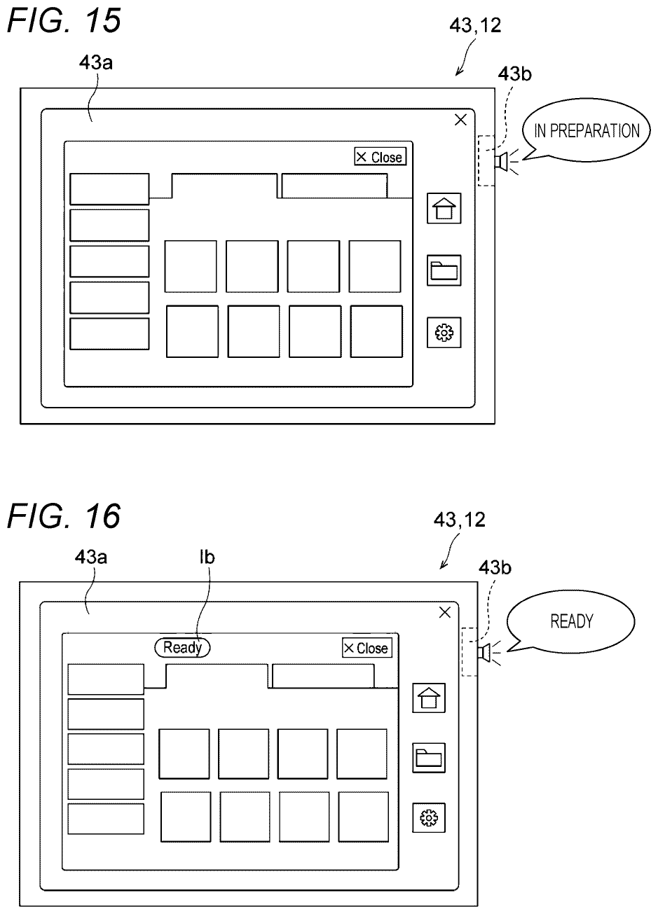

FIGS. 15 to 18 are views illustrating a fifth notification form example in the user interface 43.

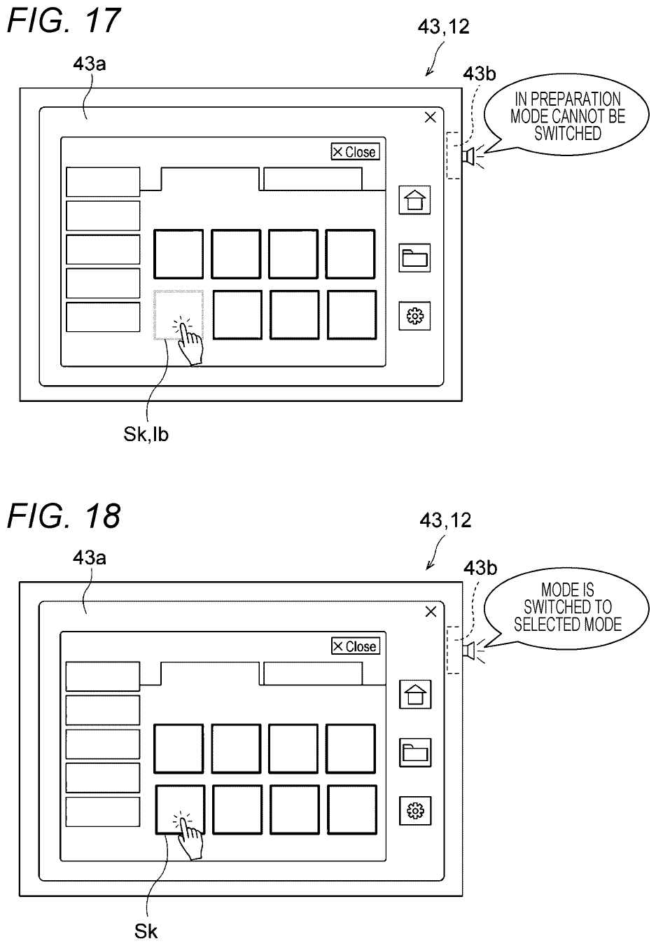

FIGS. 15 and 16 illustrate an example of a case where speech notification is combined with the first notification form example (see FIGS. 5 and 7). FIGS. 17 and 18 illustrate an example of a case where speech notification is combined with the third notification form example (see FIGS. 10 and 11).

The notification in the present notification form includes a notification by sound. The notification by sound may be emitted regardless of operation by the user or may be issued in accordance with operation by the user.

For example, as in the examples illustrated in FIGS. 15 and 16, a preparation state of the light source device 13 (that is, light sources 31 and 32) may be emitted from the speaker 43b of the user interface 43 as sound information regardless of the operation by the user. In other words, in a case where the temperature of the light source device 13 is out of the allowable temperature range, a fact that the light source device 13 is in preparation may be emitted by speech from the speaker 43b (see FIG. 15). On the other hand, in a case where the temperature of the light source device 13 reaches the allowable temperature range and the light source device 13 is ready for operation, a fact that the light source device 13 is ready may be emitted by speech from the speaker 43b (see FIG. 16).

On the other hand, as illustrated in FIG. 17, in a case where the user performs operation of selecting an unswitchable mode (that is, an unavailable mode), a fact that the mode is not switchable to the selected mode may be emitted by speech from the speaker 43b. On the other hand, as illustrated in FIG. 18, in a case where the user performs operation of selecting a switchable mode (that is, an available mode), a fact that the mode can be switched to the selected mode may be emitted by speech from the speaker 43b.

### (Modifications of Notification Form)

The above-described first to fifth notification form examples are merely examples, and other notification forms may be adopted.

For example, the notification image Ib may be displayed in a blinking manner, may be displayed in a changed color, may be displayed as a still image, or may be displayed as a moving image (animation image). The notification image Ib to be displayed in a blinking manner, in a changed color, and/or as a moving image is easily noticeable, and thus, the notification image Ib is easily recognized by the user even if the display size is small.

In addition, the notification image Ib may be displayed as a simple display image that is not selectable by the user or may be displayed as an image constituting a software key that is selectable by the user. In a case where an area of the touch panel display 43a corresponding to the notification image Ib is touched by the user, the control unit 40 may display information regarding the notification image Ib on the touch panel display 43a.

Furthermore, the sound information to be emitted from the speaker 43b may be sound having no linguistic meaning (for example, melody sound or warning sound). For example, in a case where an unswitchable (unavailable) mode is selected by the user, even if simple warning sound is emitted from the speaker 43b, the user can recognize that the mode is not switchable to the mode. The notification via the notification units 14 and 43 may include only the sound information without including the display information.

Note that the above-described notification form example is an example of the notification to be made on the user interface 43 of the control device 12, but is also applicable to the display device 14. In particular, by configuring the display 14a of the display device 14 by a touch panel display, the display 14a can also make a notification of the display information similar to the above-described notification form examples (see FIGS. 5 to 18).

### (Control Method)

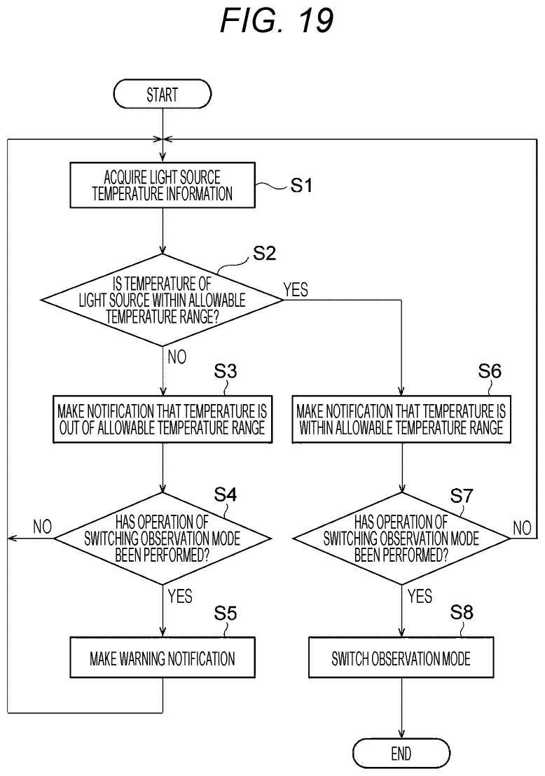

FIG. 19 is a view illustrating an example of mode switching flow in the medical observation system 10. Note that processing step S1 to processing step S8 illustrated in FIG. 19 are appropriately performed under the control of the control device 12 (particularly, the control unit 40).

In this example, in a state where the medical observation system 10 is activated, the light source temperature information D1 is acquired by the temperature detection unit 33 (see FIG. 2) (S1 of FIG. 9), and it is determined whether or not the temperatures of the light sources 31 and 32 are within the allowable temperature ranges on the basis of the light source temperature information D1 (S2). As described above, determination as to whether or not the temperatures of the light sources 31 and 32 are within the allowable temperature ranges may be performed by the control device 12 (particularly, the control unit 40) or may be performed by the light source device 13 (particularly, the temperature detection unit 33).

In a case where it is determined that the temperatures of the light sources 31 and 32 are out of the allowable temperature ranges (S2: N), the notification units 14 and 43 make a notification that the temperatures of the light sources are out of the allowable temperature ranges (S3).

Then, the control unit 40 determines whether or not the user has performed operation of switching the observation mode on the control device 12 (for example, the touch panel display 43a) (S4). In a case where the operation of switching the observation mode has been performed (S4: Y), the notification units 14 and 43 make a warning notification (S5), and the mode switching processing returns to step S1 described above. On the other hand, in a case where the operation of switching the observation mode has not been performed (S4: N), the notification units 14 and 43 do not make a warning notification, and the mode switching processing returns to step S1 described above.

On the other hand, in a case where it is determined that the temperatures of the light sources 31 and 32 are within the allowable temperature ranges (S2: Y), the notification units 14 and 43 make a notification that the temperatures of the light sources are within the allowable temperature ranges (S6). Then, the control unit 40 determines whether or not the user has performed the operation of switching the observation mode on the control device 12 (S7). In a case where the operation of switching the observation mode has been performed (S7: Y), the control unit 40 performs various kinds of control related to the mode selected by the user and switches the observation mode (S8). On the other hand, in a case where the operation of switching the observation mode has not been performed (S7: N), the mode switching processing returns to step S1 described above without the observation mode being switched. The temperatures of the light sources can vary with time depending on the situation, and thus, even if the temperatures once fall within the allowable temperature ranges, the temperatures may then fall outside the allowable temperature ranges. In the present example, as described above, in a case where the operation of switching the observation mode is not performed even after the temperatures of the light sources are once determined to be within the allowable temperature ranges (S7: N), acquisition of the light source temperature information (S1) and determination as to whether or not the temperatures of the light sources are within the allowable temperature ranges (S2) are performed again. By this means, the desired observation mode is more reliably performed under an appropriate light source temperature.

As described above, according to the above-described embodiment, the notification based on the temperature of the light source device 13 is made, and thus, the user can smoothly and accurately perform surgery, examination, research, or other medical practice. For example, the user can accurately grasp whether or not the light source device 13 is in an available state and can observe the observation target in a state where the observation target is irradiated with light emitted from the light source device 13 under an appropriate temperature condition.

In addition, in a case where the user selects an unswitchable (unavailable) mode, a notification indicating that the mode is not switchable to the mode or including the reason why the mode is not switchable to the mode is made, so that the user can appropriately and easily understand that the mode is not switchable to the selected mode. The notification according to the situation is made on the basis of the light source temperature information D1 in this manner, so that it is possible to prompt the user to quickly switch the mode to the desired mode after the light source device 13 is ready while reducing dissatisfaction of the user while the mode is not switchable to the desired mode. (Modifications)

In the above-described embodiment, the light source device 13 is connected to the rigid endoscope 18 (see FIG. 1), but the light source device 13 may be connected to any other light emission device.

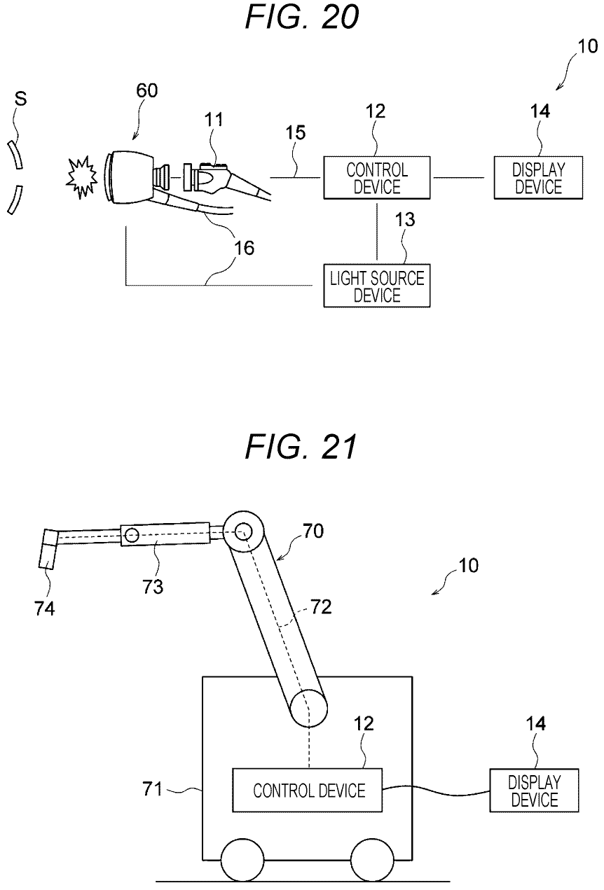

FIG. 20 is a conceptual view illustrating an example of the medical observation system 10 configured as an operative field illumination observation device in which a ring light 60 (an open field illumination device for observing biological tissues) is connected to the light source device 13.

In the example illustrated in FIG. 20, the light source device 13 and the imaging device 11 are connected to the ring light 60. The light to be emitted by the light source device 13 is sent to the ring light 60 via the light guide 16 and is emitted from the ring light 60 toward the observation target S. The ring light 60 is different from the above-described rigid endoscope 18 (see FIG. 1) in that while the rigid endoscope 18 is intended to emit light inside the observation target S and irradiate the observation target S with the light, the ring light 60 is used to emit light outside the observation target S.

In each of the above-described embodiments, in place of the rigid endoscope 18, the ring light 60 of the present example may be connected to the light source device 13 and the imaging device 11 to acquire a captured image of the observation target S and acquire an output image based on the captured image. In this case, observation light from the observation target S enters the imaging device 11 through the ring light 60, and the captured image of the observation target S is acquired by the imaging device 11. Then, the control device 12 generates an output image on the basis of the captured image acquired in this manner, and the output image is displayed on the display device 14.

Note that the light emission device that is connected to the light source device 13 and emits light does not have to be connected to the imaging device 11 and may be provided separately from the imaging device 11 (in particular, the imaging unit 52).

FIG. 21 is a view illustrating an example of the medical observation system 10 configured as a microscope system. The medical observation system 10 illustrated in FIG. 21 is a surgical microscope system having a function of capturing an image while enlarging a visual field area of the observation target and displaying an output image generated on the basis of the captured image.

The medical observation system 10 (surgical microscope system) of the present example includes a microscope device 70 that captures an image of the observation target and the display device 14 that displays the image captured by the microscope device 70. The display device 14 illustrated in FIG. 21 is provided separately from the microscope device 70, but may be provided integrally with the microscope device 70.

The microscope device 70 includes a microscope portion 74 that captures an image while enlarging a minute part of the observation target, a support portion 73 having an arm that rotatably supports the microscope portion 74, and a base portion 71 that rotatably supports the support portion 73 and is movable on a floor surface. The base portion 71 includes the control device 12 that controls operation of the medical observation system 10. The control device 12 is connected to the microscope portion 74 via a transmission cable 72. The base portion 71 may employ a configuration fixed to a ceiling, a wall surface, or the like.

The microscope portion 74 includes the above-described imaging device 11 (see FIGS. 1 and 20; not illustrated in FIG. 21), an operation portion (not illustrated) such as a switch that receives an input of an operation instruction of the microscope device 70, and a cover glass (not illustrated) for protecting the inside. The user can move the microscope portion 74 while operating the operation portion of the microscope portion 74.

In this example, the captured image of the observation target is acquired by the microscope portion 74. Then, similarly to each of the above-described embodiments, the control device 12 generates an output image from the captured image, and the output image is displayed on the display device 14.

In the microscope portion 74 of the present example, a light emission device (not illustrated) that is connected to a light source device (not illustrated) and emits light from the light source device may be provided integrally with the microscope portion 74 (imaging unit) or may be provided separately from the microscope portion 74. As an example, the light emission device may be provided on the base portion 71, may be provided on the support portion 73, or may be provided separately from the microscope device 70.

It should be noted that the embodiment and modifications disclosed herein are illustrative only in all respects and are not to be construed as limiting. The above-described embodiment and modifications can be omitted, replaced, and changed in various forms without departing from the scope and spirit of the appended claims. For example, the above-described embodiment and modifications may be combined in whole or in part, and embodiments other than the above-described embodiment and modifications may be combined with the above-described embodiment or modifications. Furthermore, the effects of the present disclosure described in the present specification are merely examples, and other effects may be provided.

The technical category embodying the above technical idea is not limited. For example, the above-described technical idea may be embodied by a computer program for causing a computer to execute one or a plurality of kinds of procedure (steps) included in a method of manufacturing or using the above-described device. In addition, the above-described technical idea may be embodied by a computer-readable non-transitory recording medium in which such a computer program is recorded. (Supplementary note)

The present disclosure can also employ the following configurations.

### Item 1

A control device including:

a temperature information acquisition unit that acquires light source temperature information that is information regarding a temperature of a light source device that emits light with which biological tissues are to be irradiated; and a control unit that controls a notification on the basis of the light source temperature information.

### Item 2

The control device according to item 1, in which the notification includes a notification regarding a state of the light source device.

### Item 3

The control device according to item 1 or 2, in which the notification includes a notification regarding a use preparation state of the light source device.

### Item 4

The control device according to any one of items 1 to 3, in which the notification includes a notification regarding availability of a function using the light source device.

### Item 5

The control device according to any one of items 1 to 4, in which the notification includes a notification regarding whether or not switching between a plurality of modes having different types of the light to be emitted from the light source device is possible.

### Item 6

The control device according to any one of items 1 to 5, in which the notification includes a notification regarding availability of the light source device. Item 7

The control device according to any one of items 1 to 6, in which the notification includes a notification by display on a display.

Item 8 The control device according to item 7, in which the notification by display on the display includes a notification to be made by activating or deactivating a software key displayed on the display.

### Item 9

The control device according to item 7 or 8, in which the notification by display on the display includes a notification to be displayed on the display by a software key displayed on the display being selected.

### Item 10

The control device according to any one of items 7 to 9, in which the display is a touch panel display.

### Item 11

The control device according to any one of items 1 to 10, in which the notification includes a notification by sound.

### Item 12

The control device according to any one of items 1 to 11, further including a notification unit that makes the notification.

### Item 13

The control device according to any one of items 1 to 11, in which the control device is provided separately from a notification unit that makes the notification.

### Item 14

The control device according to any one of items 1 to 13, in which the light source temperature information includes information regarding the temperature of the light source device.

### Item 15

The control device according to any one of items 1 to 14, in which the light source temperature information includes information as to whether or not the temperature of the light source device is within an allowable temperature range.

### Item 16

The control device according to any one of items 1 to 15, in which the control unit controls the notification on the basis of a state of the light source device evaluated by comparing the light source temperature information acquired by the temperature information acquisition unit with reference data of the light source temperature information.

### Item 17

The control device according to any one of items 1 to 16, in which the control unit controls a temperature adjustment unit that adjusts the temperature of the light source device.

Item 18 The control device according to item 17, in which the control unit controls the temperature adjustment unit to individually adjust temperatures of a plurality of light sources included in the light source device.

### Item 19

The control device according to item 17, in which the control unit controls the temperature adjustment unit to collectively adjust temperatures of a plurality of light sources included in the light source device.

### Item 20

The control device according to any one of items 1 to 19, in which the light to be emitted from the light source device includes visible light.

Item 21 The control device according to any one of items 1 to 20, in which the light to be emitted from the light source device includes excitation light that excites a specific fluorescent substance.

### Item 22

### An Observation System Including:

a light source device that emits light with which biological tissues are to be irradiated; and

a control device, in which the control device includes a temperature information acquisition unit that acquires light source temperature information that is information regarding a temperature of the light source device, and a control unit that controls a notification on the basis of the light source temperature information.

### Item 23

The observation system according to item 22, further including:

an imaging device that acquires an image of the biological tissues; and

a display device that displays the image.

### Item 24

A control method including:

acquiring light source temperature information that is information regarding a temperature of a light source device that emits light with which biological tissues are to be irradiated; and controlling a notification on the basis of the light source temperature information.

It should be understood by those skilled in the art that various modifications, combinations, sub-combinations and alterations may occur depending on design requirements and other factors insofar as they are within the scope of the appended claims or the equivalents thereof.

### REFERENCE SIGNS LIST

10 Medical observation system
11 Imaging device
12 Control device
13 Light source device
14 Display device
14a Display
14b Speaker
15 Signal transmission cable
16 Light guide
16a First light guide end portion
16b Second light guide end portion
18 Rigid endoscope
20 Insertion portion
21 Insertion distal end portion
22 Optical connection portion
23 Imaging connection portion
31 First light source
32 Second light source
33 Temperature detection unit
34 Temperature adjustment unit
40 Control unit
41 Temperature information acquisition unit
42 Image processing unit
43 User interface
43a Touch panel display
43b Speaker
44 Storage unit
60 Ring light
70 Microscope device
71 Base portion
72 Transmission cable
73 Support portion
74 Microscope portion
D1 Light source temperature information
D2 Captured image data
D3 Output image data
Ia Captured image
Ib Notification image
S Observation target
Sk Software key

1. A control device comprising:
a temperature information acquisition unit that acquires light source temperature information that is information regarding a temperature of a light source device that emits light with which biological tissues are to be irradiated; and
a control unit that controls a notification on a basis of the light source temperature information.

2. The control device according to claim 1,
wherein the notification includes a notification regarding a state of the light source device.

3. The control device according to claim 1,
wherein the notification includes a notification regarding a use preparation state of the light source device.

4. The control device according to claim 1,
wherein the notification includes a notification regarding availability of a function using the light source device.

5. The control device according to claim 1,
wherein the notification includes a notification regarding whether or not switching between a plurality of modes having different types of the light to be emitted from the light source device is possible.

6. The control device according to claim 1,
wherein the notification includes a notification regarding availability of the light source device.

7. The control device according to claim 1,
wherein the notification includes a notification by display on a display.

8. The control device according to claim 7,
wherein the notification by display on the display includes a notification to be made by activating or deactivating a software key displayed on the display.

9. The control device according to claim 7,
wherein the notification by display on the display includes a notification to be displayed on the display by a software key displayed on the display being selected.

10. The control device according to claim 7,
wherein the display is a touch panel display.

11. The control device according to claim 1,
wherein the notification includes a notification by sound.

12. The control device according to claim 1, further comprising:
a notification unit that makes the notification.

13. The control device according to claim 1,
wherein the control device is provided separately from a notification unit that makes the notification.

14. The control device according to claim 1,
wherein the light source temperature information includes information regarding the temperature of the light source device.

15. The control device according to claim 1,
wherein the light source temperature information includes information as to whether or not the temperature of the light source device is within an allowable temperature range.

16. The control device according to claim 1,
wherein the control unit controls the notification on a basis of a state of the light source device evaluated by comparing the light source temperature information acquired by the temperature information acquisition unit with reference data of the light source temperature information.

17. The control device according to claim 1,
wherein the control unit controls a temperature adjustment unit that adjusts the temperature of the light source device.

18. The control device according to claim 17,
wherein the control unit controls the temperature adjustment unit to individually adjust temperatures of a plurality of light sources included in the light source device.

19. The control device according to claim 17,
wherein the control unit controls the temperature adjustment unit to collectively adjust temperatures of a plurality of light sources included in the light source device.

20. The control device according to claim 1,
wherein the light to be emitted from the light source device includes visible light.

21. The control device according to claim 1,
wherein the light to be emitted from the light source device includes excitation light that excites a specific fluorescent substance.

22. An observation system comprising:
a light source device that emits light with which biological tissues are to be irradiated; and
a control device,
wherein the control device includes
a temperature information acquisition unit that acquires light source temperature information that is information regarding a temperature of the light source device, and
a control unit that controls a notification on a basis of the light source temperature information.

23. The observation system according to claim 22, further comprising:
an imaging device that acquires an image of the biological tissues; and
a display device that displays the image.

24. A control method comprising:
acquiring light source temperature information that is information regarding a temperature of a light source device that emits light with which biological tissues are to be irradiated; and
controlling a notification on a basis of the light source temperature information.
